(19) United States
(12) Patent Application Publication
(10) Pub. No.: US 2010/0112659 A1
(43) Pub. Date: May 6, 2010

(54) FERMENTATION PROCESS

(76) Inventor: Daniel R. DeBrouse, Wellston, OK (US)

Correspondence Address: DUNLAP CODDING, P.C., PO BOX 16370, OKLAHOMA CITY, OK, 73113, US

(21) Appl. No.: 12/644,836

(22) Filed: Dec. 22, 2009

Related U.S. Application Data
(63) Continuation of application No. 11/809,746, filed on Jun. 1, 2007, now abandoned.
(60) Provisional application No. 60/810,237, filed on Jun. 2, 2006.

(51) Int. Cl. C12P 1/04; C12P 1/02

(52) U.S. Cl. 435/170; 435/171

(57) ABSTRACT

A process for the biosymbiotic fermentation of one or more microbe species selected for production of bioactive molecules. These microbe species (bio-hosts) are fermented together with both botanical sources or bacterial, fungal or algal organisms (growth-symbiots) as promoter stimulants. In this manner yields of the bioactive molecules equivalent to those obtained through the biotechnological generation of expression vectors are possible. Furthermore, the invention contemplates biodegradation processes for the decomposition of the resulting fermentation biomass in such a way as to concentrate the bioactive molecules of interest in a precipitate or oleoresin form.

### CROSS-REFERENCE TO RELATED APPLICATIONS

The present application is a continuation of U.S. Ser. No. 11/809,746, filed Jun. 1, 2007, which claims the benefit under 35 U.S.C. 119(e) of U.S. Provisional Application Ser. No. 60/810,237, filed Jun. 2, 2006, the entire disclosures of both applications are hereby expressly incorporated herein by reference.

### STATEMENT REGARDING FEDERALLY SPONSORED RESEARCH OR DEVELOPMENT

Not applicable.

### BACKGROUND OF THE INVENTION

There is no known previous art of biosymbiotic fermentation within the biotechnology industry, however. Microbiologists have established, for example, the interlinking-nutrient cooperations between microbes within a local ecosystem. Such interactions have at their molecular bases a genetic response to the presence of a biological “marker” from a local resident or transit organism. Another type of microbial symbiosis occurs within the mammalian intestine. In this nutrient rich environment microorganisms can colonize and grow, resulting in numerous interactions between microorganisms and the animal host which impact the health and well being of the host.

In yet other types of symbioses, microbes respond to the presence of transit organisms in an alerted state of proliferation. For example, the group of pigmented yeasts, the xanthophyllomyces, are known to enter a hypersensitive state of carotenoid production in response to the presence of particular fungi. In the present invention, the symbiotic interactions are in a similar category as those types observed in this xanthophyllomyces example. In a diverse community, microorganisms may be symbiotically stimulated to express elevated levels of agents having pharmacologically or nutritive importance and to naturally excrete concentrated forms of such agents through biodegradation processes which have evolved in nature.

### BRIEF DESCRIPTION OF THE DRAWINGS

FIG. 1 shows a perspective view of an apparatus for Vertical Gas Mediated Extraction (VGME) which is used in certain embodiments of the present invention.

### DETAILED DESCRIPTION OF THE INVENTION

The present invention in one embodiment involves identification of a bio-host such as a plant, fungus or microbe which is a source of a desired class of biologically active molecules including, but not limited to, lipid soluble vitamins and pigments. For example, in the case of the carotenoid lycopene, a botanical source of lycopene such as Lycopersicum esculentum (tomato), or a prokaryotic source such as a species belonging to the genus Paracoccus may be the bio-host.

The identity of the bio-host predetermines the subsequent procedures in a biosymbiotic fermentation. Therefore, from the example above involving Lycopersicum esculentum as the bio-host, the biosymbiotic fermentation process involves a biosymbiotic decomposition process, which, in general, comprises the microbiological or natural “detergent” decomposition of a biomass in such a manner as to bring about the concentration of a particular biologically active molecule in a precipitate or oleoresin form derived therefrom. Where used herein the term “bio-host” may be a living organism or a material derived from a living organism, either of which may comprise a source, or enhanced source of the bioactive molecule of interest.

In another example, if a bacterial species is chosen instead of a botanical biomass, one next identifies a growth-symbiot comprising or able to express a stimulant capable of effecting an elevated expression of the biologically active molecule in the bio-host organism. Such a growth-symbiot may comprise one or more fungal, bacterial or viral growth-symbiots entirely capable of continued proliferation throughout the course of the fermentation cycle, or a biological “marker” or “inducer” capable of transducing a biochemical signal within a host or some general transcriptional control over the bio-host promoter region(s) within the operon or gene encoding a particular biologically active molecule. Where used herein the term “growth-symbiot” may refer to a living organism such as a bacterium, alga, or fungus, or may refer to an organic composition or substance which induces or enhances production of a bioactive molecule or interest.

Upon the identification of a viable bio-host and the one or more growth-symbiots, a nutrient broth is formulated for the optimal propagation of the symbiot(s) throughout the course of the fermentation cycle. In general the nutrient medium will contain an appropriate carbon source in addition to minerals, salts, growth factors and other components common to those skilled in the art of microbial fermentation. The fermentation period, temperature, pH and carbon dioxide levels and the like are selected in part through literature review and symbiot microbial tolerance and biochemical panel testing.

At the end of the fermentation period of the bio-host and the growth-symbiot, except in the case of some biosymbiotic decomposition processes, the molecular goal from this point on is to isolate the pool of biologically active molecule. In practice this is done using one or any combination of three solvent-free extraction procedures, as described below in regard to the previous example of lycopene biosymbiotic fermentation.

First, when the tomato Lycopersicum esculentum is the bio-host source of lycopene (as derived from the tomato fruit), upon completion of the biosymbiotic decomposition period one may enzymatically lyse the growth-symbiots in addition to any remaining tomato fruit cells, for example by using any of a number of commercially available enzymes such as lysozyme from eggs and papain from papaya fruit.

Second, in place of enzymatic lyses, a detergent can be added to solubilize lipids, starches and other complex macromolecules. A preferred detergent would be one comprising of a concentration of fatty acids, papain, and salt. Next, a volume of sodium alginate gel can be added to dehydrate the fermentation solids into beadlet form through cold temperature spray atomization into a cloud of cornstarch collectant. Upon separation of the cornstarch from the fermentation beadlets, the beadlets can be then dispersed into a small volume of a hydrated form of commercially available fatty acid and finally subjected to a third extraction process, Vertical Gas Mediated Extraction (VGME), for yields of lycopene ranging in purity levels for example from 5 to 99 percent. Vertical Gas Mediated Extraction is another technology unique to the present invention.

The invention as described and contemplated herein is not limited in its application by the details of construction and the arrangements of the components set forth in the present examples or illustrated in the drawings. The invention is capable of other embodiments or of being practiced or carried out in various ways. Also, it is to be understood that the phraseology and terminology employed herein is for the purpose of description and should not be regarded as limiting.

The present invention relates in one embodiment to the biosymbiotic fermentation of microorganisms for the harvest of pharmacologically active (bioactive) agents, preferably at industrial yields. The technology typically involves a molecular response within the promoter region of a particular operon present in the chromosome of one microbe in response to a biochemical “marker” or “inducer” presence at concentrations sufficient to signal the transcriptional control pathway of a particular set of genes. For example, this phenomenon was observed by cross contamination of the pigmented yeast Phaffia rhodozymas S345-1 (the bio-host in this example) with the mold Penicillium citrinum S244-1 (the growth-symbiot in this example). As a result of the rate of proliferation of the mold, signaled through the secretion of specific biochemical metabolic markers, the pigmentation of the yeast darkened dramatically within the first 18 hours of nutritional competition. Upon HPLC analysis of the fermentation solids, we found an elevation in the levels of the carotenoid astaxanthin which was 19× that found under mono-cultural (yeast only) conditions.

The bio-host organism of choice for the establishment of a biosymbiotic fermentation process need only be limited by the identification of a growth-symbiot organism or some natural biomolecule to effect the desired elevations, or extractions of the desired bioactive molecule. Pigmented bioactives are typically more readily obtained in yields exceeding 10% of the dry fermentation solids weight, than that of non-pigmented bioactive molecules. However, in accordance with the invention, it is possible to symbiotically signal the transcriptional controls operating the expression of any number of fermentable pharmacological (bioactive) agents.

In the practice of this first step in the fermentation cycle one first identifies a bio-host organism carrying a genetic trait encoding some transcript or family of transcripts of some pharmacologic or biological importance or value or, alternatively an eucaroytic botanical reservoir of the molecules, such as a vegetative or fruit source. To better demonstrate the basic principles thus far known in biosymbiotic fermentation processes, the example referenced above, regarding the carotenoid lycopene can be taken.

To proceed, an understanding of the fundamental chemical and physical properties of the lycopene molecule is desired as well as insight into the molecular basis of the particular class of molecules lycopene belongs to and its natural reservoirs. Lycopene is an acyclic open-chain unsaturated polyene hydrocarbon presenting 13 carbon-carbon double bonds of which 11 are conjugated in a linear array. Lycopene exists predominantly in nature in its most thermodynamically stable all-trans isomeric form. However, about 7 of the conjugated bonds in its all-trans form may isomerize from trans to mono- or poly-cis forms. In mammals lycopene is believed to be of nutritional value through its behavior as a powerful antioxidant, quenching highly reactive singlet oxygen and trapping peroxyl radicals. In botanicals, however, lycopene belongs to a vital family of some 600 known photosynthetic pigments classified as carotenoids. Carotenoids are widely distributed in nature being involved in the capture of light energy in the process of photosynthesis.

The carotenoids range in color from red, as in the case of lycopene, to orange, pink and yellow and are found within all chloroplasts. In chloroplasts carotenoids are always found in association with chlorophyll a. In green leaves the color of the carotenoids is masked by the much more abundant chlorophylls. In other tissues, however, red tomato fruits for example, the carotenoids predominate. Carotenoids are also present in the prokaryotic blue-green algae, the bacterial family of Enterobacteriaceae, and within the bacterial genera Pantoea and Paracoccus, where they are believed to function in the protection of the cell against photooxidative damage. Like the chlorophylls, carotenoid chloroplast pigments of botanical cells are embedded in the thylakoid membranes, protein bound. In prokaryotic cells carotenoids may be found in association with membrane proteins. Carotenoids, being lipid soluble, are rarely found free within the cytoplasm of cells.

Lycopene is naturally biosynthesized from phytoene through four sequential dehydrogenation reactions through the removal of eight hydrogen atoms, catalyzed by a gene encoding phytoene desaturase. Intermediates of this reaction include phytofluene, carotene and neurosporene. Furthermore, all carotenoids in nature are derived from the isoprene biosynthetic pathway and its five carbon building block, isopentenyl pyrophosphate (IPP).

Without wishing to be bound by theory, it is useful to understand the basic molecular knowledge of how bio-hosts of the present invention may accumulate lycopene (or other bioactive molecules), primarily in the case of bacterial and fungal bio-hosts, particularly in regard to the fundamental controls of gene expression.

As well understood by persons of ordinary skill in the art, cells have evolved an elaborate regulatory system to determine which of their thousands of genes to transcribe at any given time. This regulatory system functions as a switch able to turn a gene on or off. The transcription of a gene, is generally controlled by a regulatory region of DNA near the site where transcription begins. Some regulatory regions are mono-signal dependent while others are complex responding to a variety of signals and switching neighboring genes on and off. Whether simple or complex such transcriptional controls consist of two fundamental types of components; short stretches of DNA of defined sequence and gene regulatory proteins that recognize and bind to them, thus serving to turn a gene on or off.

In the case of a bacterial bio-host it is at these transcriptional control regions of the codons of the bioactive molecule, to which is indirectly delivered a transcription start signal to the regulatory region of the gene of interest. In bacterial bio-hosts such signals are usually delivered through a fungal biological “marker” or “inducer”.

Therefore, in the case of the use of the bacterium Paracoccus marcusii as bio-host for the biosymbiotic fermentation of lycopene, this organism both possesses and regulates the expression of a carotenoid biosynthetic pathway.

In nature the bacterium activates the carotenoid biosynthetic pathway in response to either light, heat or from the presence of perhaps one or more classes of inorganic, organic or other biological molecule in its local environment. The Paracoccus bacterium uses a positive control type of gene regulation and the regulatory protein component of these genetic switches are referred to as transcriptional or gene activator proteins.

The Paracoccus transcriptional activator operates in much the same manner as a light switch to turn gene expression on or off. For example, the Paracoccus transcriptional activator protein may activate the carotenoid biosynthetic set of genes in response to a simple depletion or presence of a ligand or inducer or inhibitor in the production of lycopene such as phytoene. Therefore in an embodiment comprising a growth-symbiot which has the trait of biosynthesizing and excreting phytoene into the fermentation medium, the Paracoccus organism may respond by switching on the carotenoid biosynthetic genes with an initial expression of the enzyme phytoene desaturase which catalyzes the biosynthesis of lycopene through four sequential dehydrogenation reactions. In this embodiment, carotenoid expression can be turned on or off through the addition of growth-symbiots to the fermentation medium. Organisms being used as bio-hosts in a biosymbiotic fermentation as described herein are preferably first studied within its natural environment noting key changes in bioactive concentrations in response to the presence of natural stimulus.

Examples of known organism and extracts thereof that are useful as growth-symbiots in a biosymbiotic fermentation are shown in Table 1.

TABLE 1
Growth Symbiots

Penicillium citrinum S244-1 and Extracts Thereof
Extracts of Citrus Fruits and Mixtures thereof such as orange,
grapefruit, lemon and lime.
Acids such as those produced from the lactic acid bacteria.

In the case of botanical bio-hosts (e.g., plant biomass sources of bioactive molecules) we are concerned only with the intercellular functions and synthesis of a particular biologically active molecule, for example how and where it functions within the cell. This information is useful since it will likely be our desire to carry out a symbiotic decomposition fermentation or natural detergent extraction of the botanical source (e.g., tomato fruit) to concentrate the bioactive of interest.

Another aspect of the establishment of a biosymbiotic fermentation in accordance with the present invention is to identify a growth-symbiot. A suitable growth-symbiot may be defined as any fungus or microbe, or natural or synthetic molecule capable of signaling its competitive intentions or expressing a particular set of decomposition properties suitable or causing induction of genes for the isolation of a particular bioactive from a biological source (the bio-host). Depending upon the bioactive molecule sought, it is possible to maintain several such fermentations in a complex population within the same vessel. For example the simultaneous symbiotic fermentation production of zeaxanthin, lycopene and lutein, provides yields of 1-6%; 1-8%; and 1-7% by weight respectively, in one embodiment.

Furthermore, when botanical biomass sources (e.g., tomato fruit) of the like bioactives are available, these can be used not only as additional bioactive source but as nutritional sources for the growth-symbiots as well. Under some scenarios the botanical matrix components, specifically the lipid fractions, may be used as encapsulation material to effectively protect the biologically active molecules from isomerization and oxidation and thus stabilizing them in an amorphous state for disintegration within the mammalian intestine for a more efficient deliver of the bioactive.

For a particular bio-host and growth-symbiot combination, a nutritional medium is preferably provided with the optimization of propagation of the growth-symbiot in mind. Standard microbiological growth media may be initially considered.

Preferably to identify such a nutritional medium, the nutritional and growth factors required for a sustained proliferation of the growth-symbiots throughout the course of the fermentation process is taken into account. Thus, we would expect to identify suitable sources of carbon, nitrogen and inorganic salts as well as any required growth factors such as vitamins and minerals. It is preferable, rather than using commercially available raw ingredients to formulate our mediums, to use a natural bio-nutrient cake, which is another novel aspect of this invention.

In principal, a bio-nutrient cake may be defined as any naturally occurring botanical biomass, including marine botanicals, such as those of fruits and vegetables or some mix thereof. Table 2 provides a list of some acceptable bio-nutrient cake, sources and components.

TABLE 2
Raw bio-Nutrient Cake Compositions

| 1. Corn, cabbage, beans, peppers, all commonly known fruits | |
|---|---|
| and vegetables. | |
| 2. Various species of botanicals, trees and extracts thereof | |
| 3. Bacteria | |
| Bacillus sutbilis | |
| B. licheniformis | |
| B. pumilius | |
| Leuconostoc mesenteroides | |
| Enterococcus faecium | S2124-1 |
| Enterococcus faecalis | S2124-2 |
| Lactobacillus acidophilus | S345-1 |
| Lactobacillus lactis | S345-2 |
| Lactobacillus delbrueckii | S345-3 |
| Lacotbacillus leichmanni | S345-4 |
| Lactobacillus salivarius | S345-5 |
| Streptococcus bovis | S2134-1 |
| Streptococcus thermophilus | S2134-2 |
| Lactobacullus bavaricus | S6114-2 |
| Lactobacillus casei | S6114-3 |
| Lactobacillus coryniformis | S6114-4 |
| Lactobacillus curvatus | S6114-5 |
| Lactobacillus plantarum | S6114-6 |
| Lactobacillus sake | S6114-7 |
| Lactobacillus brevis | S845-1 |
| Lactobacillus buchneri | S845-2 |
| Lactobacillus cellobiosus | S845-3 |
| Lactobacillus confusus | S845-4 |
| Lactobacillus coprophilus | S845-5 |
| Lactobacillus fermentatum | S845-6 |
| Lactobacillus sanfrancisco | S845-7 |
| Leuconostoc dextranicum | S845-8 |

Bio-nutrient cakes may be manufactured through the dehydration of concentrated pastes of the vegetative or fruit biomasses and mixes thereof. For example, referring to the earlier lycopene example using Paracoccus mucassi S286-1 as bio-host and a standardized steam extract of orange, grapefruit and lemon peel as growth-symbiot. Numerous botanical sources of lycopene were identified. The botanical sources of lycopene, such as the tomato fruit, to possesses a healthy pool of phytonutrients beneficial to a sustained growth of the Paracoccus bio-host in addition to abundant carbon, nitrogen, salts, minerals and amino acids.

In this particular example, tomato fruit may be obtained commercially as a concentrated paste. However, to demonstrate the technique, 10 pounds of tomato fruit may be taken and ground to a puree and then concentrated to a paste at 100° C. under constant agitation. Next a small volume of emulsifier such as one consisting 70% olive oil, 29.5% palmitic acid and 0.5% myristic acid is added. The mixture is agitated to a flowing paste and spray-dried. 77 g of this dehydrated tomato bio-nutrient would be used per 1 liter of fermentation medium.

It is important to note a bio-nutrient cake may be prepared from two or more botanical biomasses in some cases. Bio-nutrient mediums may be sterilized prior to inoculation as with any other microbiological medium.

Once formulated and sterilized the fermentation medium can be inoculated with a bacterial load of the bio-host, e.g., of 3.7 million bio-host cells (e.g., Paracoccus marcusii S286-1) per 1 liter of fermentation medium.

At the end of the fermentation period it is desired to isolate the pool of biologically active molecules. In practice one or some combination of 3 solvent-free extraction procedures can be chosen, although the possible extraction procedures are not limited to these three processes.

The first of these, enzymatic decomposition, is preferably used as a first processing step after the completion of a biosymbiotic fermentation. In this case the fermentation solids are collected through filtration methods commonly known to those in the brewery industry. Once collected the fermentation solids are dissolved in a volume of water containing, for example, 10 g/1 L papaya and 5 g/1 L of sodium chloride in addition to other suitable enzymes such as lysozyme. Again the fermentation solids would be collected by filtration and washed 3 times with distilled water.

Next a volume of the emulsifying lipid oil introduced earlier may be added at a ratio of 50 mL/160 g of fermentation solids. This mixture is then agitated to a flowing foam consistency. At this stage a large volume of water can be added and the lipid soluble bioactive molecule could be oiled or precipitated out of solution. Alternatively the foam end product of this second extraction step may be processed in a more specific and refined extraction procedure, Vertical Gas Mediated Extraction (VGME).

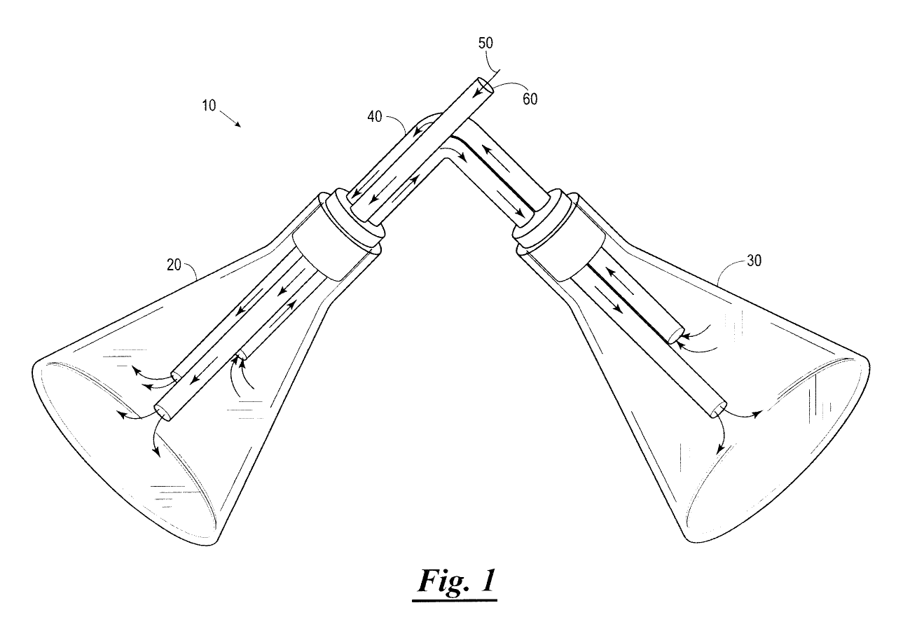

VGME is most beneficial to the extraction of lipid soluble molecules from a solubilized foam of the lipid containing matrix. A VGME unit 10 (FIG. 1) comprises at least two vessels 20 and 30 attached in a loop 40 through which a carrier gas 50 may flow. The extraction vessel 20 comprises a gas inlet 60 which is used to charge extraction vessel 20 with the carrier gas 50. It is within this extraction vessel 20 that the carrier gas 50 first contacts and picks-up a lipid extract. Upon picking up a molecule of lipid the carrier gas 50 may then travel to the receiving vessel 30 within which the bioactive is deposited. Alternatively, the carrier gas 50 may be allowed to flow through a column of calcium alginate packed gel for the separation of closely related lipids.

The objective in conducting a successful VGME is to establish a vaporous suspension or “cloud” within the extraction chamber that may completely interact with the extraction foam in either a static or dynamic state of flow. Suitable carrier gases include, but are not limited to, butane, carbon dioxide and in some cases inert gases such as helium.

Finally, in some cases, such as if the tomato Lycopersicum esculentum was chosen as the bio-host source of lycopene, a bio-decomposition symbiotic fermentation process could be followed which would result in the digestion of 80-88% of the vegetative biomass. Here the fermentations are preferably carried out at 25-42° C.

Preferred anaerobic and aerobic biodecomposition growth-symbiot organisms contemplated for use herein may comprise at least one of Bacillus subtilis S4115-1, Bacillus lichenifonnis S4125-2, Bacillus pumilius S494-1 and species belonging to the genus Lactobacillus (e.g., see Table 2). The key to this type of biosymbiotic fermentation is the selection of decomposition organisms that are able to effect the decomposition of both the botanical bio-host and/or any fungal or microbial populations present within the vessel. Preferably, in the practice of this technology, through the correct selection of organisms, an amphibolism decomposition cycle may be run. For this mechanism, the particular set of decomposition organisms is selected from soil saprobes that degrade cellulose, lignin, and other complex macromolecules. In addition to the decomposition organisms, sometimes it is beneficial or necessary to add concentrations of mineral salts, such as sodium chloride, and detergents, ranging in levels from 0-33% of the crude biomasses preferred detergents include, for example, sodium lauryl sulfate. However, many acceptable commercial detergents are available. Furthermore, it is sometimes beneficial to add degradative enzyme pools, in their natural states, such as those profiles found in natural pineapple juice and in meat tenderizers presently used within the food industry.

At the end of the bio-decomposition period the fermentation biomass should have been reduced by 80-88%. By the end of the decomposition process the majority of the decomposition organism pool should have lysed due to the depletion of the nutrient source. At this stage we may extract the desired bioactive through extraction methods 2 and 3 outlined previously.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples, which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLES

### Example 1

### Biosymbiotic Fermentation to Obtain Lycopene

Ten pounds of commercially available tomato paste was spray dried to 6 pounds of concentrated tomato powder according to the following method; 2 gallons of distilled de-ionized water containing 10 g of commercially available papain and 500 mL of commercially available concentrated pineapple juice was stirred into the 10 pounds of tomato paste. The mixture was agitated for a period of 15 minutes prior to the infusion of 500 g of a sodium alginate gel (gel was made by adding 127 g of sodium alginate to cold water under gentle agitation). After 5 additional minutes of agitation the mixture was spray dried according to methods commonly known to those skilled in the art. This method yielded the 6 pounds of concentrated tomato powder. One liter of isoprene support medium was prepared by mixing 77 g of the concentrated tomato powder together with 3.5 g of commercially available molasses and 1.5 g of corn steep liquor in a 1 L fermentation vessel. This mixture was diluted to 1 liter with distilled de-ionized water containing 360 ppm citrus extract growth-symbiot (as prepared through the steam distillation of the following citrus peels; orange, grapefruit and lime, the essential oils were collected in a receiving flask and used as is without further processing). The medium was then autoclaved at 121° C. for a period of 15 minutes and then allowed to cool to room temperature prior to inoculation with 3.7 million cells of Paracoccus marcusii S286-1 as the incubated without stirring for a period of 18 hours at 26° C. A red solid precipitate appeared on the surface of the medium about the 16 hours after the start of incubation. At the 18 hour mark the red solid was collected by vacuum filtration and washed 3 times with cold water. The crude solid was next dried under vacuum in the dark at 37° C. in the dark. 1.8 g of red powder was yielded from this process which uses confirmed to contain 32.3% lycopene.

### Example 3

### Biosymbiotic Fermentation to Obtain Astaxanthin

1 liter of growth-symbiot potato medium was made by blending 100 g or raw whole potatoes, including skin, 1.5 g of corn steep liquor and 20 g of dextrose in 1 liter of de-ionized distilled water. The broth was sterilized at 121° C. for a period of 15 minutes. The medium was allowed to cool to room temperature prior to the addition of 1 mL of a growth-symbiot comprising a standardized extract of the mold penicillium (prepared by adding 5 g/L agar to the above formula), sterilizing and pre-pouring 10 ml of the hot post sterilized medium into a sterile petri dish and allowing it to solidify prior to streaking with a loop full of Penicillium. The plate was incubated at 26° C. for a period of 72 hours. The contents of the growth plate was blended into 500 mL of distilled de-ionized water and filter sterilized under vacuum. 1 mL of this solution was then added to the above broth. The medium comprising the potato and Penicillium extract was then inoculated with the bio-host yeast Phaffia rhodozyma S345-1 bio-host organism. The biosymbiotic fermentation was carried out at 26° C. for a period of 48 hours with one pH adjustment, to 7.2 with 10% sodium hydroxide solution, after the first 24 hours of incubation. At the end of the fermentation period the fermentation solids were collected by filtration and dissolved in 250 mL of distilled de-ionized water containing 5 g of sodium chloride and 0.5 g of polyoxyethylene (20) sorbitan as commercially available.

The resultant mixture was agitated gently for a period of 15 minutes. The mixture was next placed in the extraction vessel of the VGME unit (the receiving vessel of the unit is charge with 70:29.5:0.5% olive oil, palmitic and myristic acid respectively) and placed under a butane flow for a period of 60 minutes. At the end of the 60 minute period, approximately 35 g of a dark red oil was collected in the receiving vessel. HPLC analysis of the red oil confirmed it to contain a concentration of 10% lycopene.

### Example 2

### Biodegradation Biosymbiotic Fermentation to Obtain Lycopene

The process is carried out as in Example 1 up to the addition of the bio-host organism, wherein at the completion of the fermentation cycle the pH of the medium is adjusted to 7.2 and the medium was inoculated with 1.7 million cells of Phanerochaete chrysosporium S6116-1 and 700,000 cells of Pseudomonas medocina S616-1 as bio-host organisms. The mixture was then as an inoculation load of 7.7 million cells. The fermentation was carried out at 26° C. for a period of 96 hours. At the end of the fermentation period the fermentation solids were collected and dissolved in 250 mL of a 99.1% mixture of distilled de-ionized water and polyethylene (20) sorbitan respectively. 5 g of sodium chloride was added and this mixture atitated to a foam. The fermentation foam was then transferred to the extraction chamber of the VGME unit and extracted under of flow of butane gas for a period of 30 minutes. 1.787 g of a pink solid was collected on the walls of the receiving chamber. The solid was found to contain 98% astaxanthin upon HPLC analysis.

### Example 4

### Biosymbiotic Fermentation to Obtain Lutein

In late 2005 we isolated a bacterium most closely related to Paracoccus mucassi. However this organism predominantly expresses the carotenoid lutein in response to depleting nutrients and upon exposures to extremes of light. This organism was isolated from Oklahoma soil on a nutrient agar plate and labeled Symbiot S785-1. This species of bacterium, as with all others listed in this document are available from our in-house library. This organism was used in the following fermentation process.

Support medium was made according to the following formula;

Grams/1 liter:

| | |
|---|---|
| Ground Soy Beans | 15.0 (dehydrated whole beans were |
| | milled to a fine powder) |
| Corn-steep Liquor | 01.0 Commercially Available |
| Glucose | 10.0 Commercially Available |

1 liter of Lutein support medium was prepared according to the above formula. The broth was sterilized at 121° C. for a period of 15 minutes. Upon cooling to room temperature, 3.2 mL of Standardized Penicillium Extract (as prepared in Example 3) was added to the medium. The broth was then inoculated with 10 mL of a load of Symbiot S785-1 equal to 3.4 million CFU/mL and incubated under gentle agitation for 15 minutes every 4 hours in the presence of light at 27° C. for a period of 72 hours. At the end of the fermentation period the fermentation solids were collected by methods commonly employed within the brewery industry. The fermentation solids were dissolved in 300 mL of distilled de-ionized water containing 1.0 g of sodium lauryl sulfate and 2 g of sodium chloride. The mixture was agitated to a foam and extracted under a flow of butane in the VGME unit (FIG. 1) for a period of 30 minutes. 0.98 g of a yellow solid was collected and showed to contain 97° k lutein.

### Example 5

### Degradation Biosymbiotic Fermentation to Obtain Lutein from Raw Spinach

2 kg of raw spinach was blended into 3 liters of cold distilled de-ionized water containing 5 g of papain and 25 mL of concentrated pineapple juice. This mixture was blended for a period of 10 minutes prior to the infusion of 3.5 g of sodium alginate. The mixture was then agitated gently for an additional 10 minutes prior to spray drying (the solution was atomized at room temperature into a swirling bed of cornstarch collectant). The collectant was gathered immediately and sifted by methods commonly known to those in the industry. 986 g of spinach beads were obtained.

250 g of the spinach bio-nutrient beads were then blended into 1 liter of distilled de-ionized water in a 2 liter fermentation vessel and inoculated with 5 mL of inocula providing a load of 1.7 million CFU/mL of Bacillus subtilis S4115-1 and 3.3 mL of inocula providing a load of 347,000 CFR/mL of Bacillus licheniformis S4125-2 as growth-symbiots. The mixture was fermented for a period of 24 hours at 27° C. with 5 minutes of gentle agitation every 6 hours. 23 hours into the fermentation 1.2 g of sodium chloride and 2 g of calcium chloride were added under gentle agitation and a yellow precipitate began to form on the surface of the medium. At the end of the 24 hour fermentation cycle the yellow precipitate was collected by vacuum filtration and dried at 37° C. n the dark under vacuum for a period of 6 hours. This process yielded 578 mg of a yellow solid which contained 86.3% lutein upon HPLC analysis.

### Example 6

### Poly Biosymbiotic Fermentation to Obtain Lycopene, Lutein and Zeaxanthin

A support medium was made by blending 3 kg of raw yellow corn kernels in 3.5 liters of water to a viscous cream. Next 2.7 g of sodium alginate was added and the mixture was agitated for a period of 15 minutes prior to spray atomizing into a swirling bed of cornstarch collectant. 1.2 kg of bio-nutrient corn beadlets were obtained from this method.

1 liter of support medium was then prepared according to the following formula;

Grams/1 liter:

| | |
|---|---|
| 250 g | Corn Bio-Nutrient |
| 4.6 g | Tomato Bio-Nutrient as prepared in example 1 |
| 7.2 g | Commercially Available Soya Powder |
| 3.0 g | Glucose. |

The medium was heated at 100° C. for a period of 2 minutes with constant stirring and then allowed to cool to room temperature.

The fermentation medium was then inoculated with the following bio-host organisms at the following loads;
3 mL of a culture of Paracoccus marcusii S286-1 providing 1.7 million CFU/mL;
1 mL of a culture of Symbiot S785-1 providing 3.2 million CFU/mL;
2 mL of a culture of Bacillus natto S-484-1 providing 2.8 million CFU/mL; and
2 mL of a culture of Bacillus pumiliums S-494-1 providing 1.8 million CFU/m L.

The medium was then fermented at 27° C. for a period of 72 hours under constant agitation. The pH of the medium was adjusted 7.2 with 10% sodium hydroxide solution every 24 hours. At the end of the fermentation period 2 mL of commercially available lysozyme was added to the medium in addition to 10 g of sodium chloride and 3 g of calcium chloride. This mixture was stirred vigorously for a period of 15 minutes prior to the addition of 3 g of sodium lauryl sulfate. The resultant mixture was agitated to a foam and transferred to the VGME unit. The receiving vessel of the unit was charged with 50 mL of an oil having the following composition; 70% olive oil, 29.5% palmitic acid and 0.5% myristic acid. The fermentation foam was extracted under of flow of butane for 60 minutes. At the end of the extraction process, 50 mL of an intensely orange oil was recovered. HPLC analysis confirmed the following composition:

| | |
|---|---|
| Lycopene | 4.6%, |
| Lutein | 2.7%, |
| Zeaxanthin | 1.2%. |

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, compositions of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A fermentation method for producing a biomolecule comprising:
providing an isolated first biological species capable of producing the biomolecule;
providing an isolated second biological species for supporting growth of the first biological species and expression of the biomolecule therein;
producing a fermentation biomass containing the biomolecule by co-fermenting the first biological species and the second biological species in a growth medium thereby causing production of the biomolecule by the first biological species, wherein production of the biomolecule is greater when the first biological species and second biological species are co-fermented than when the first biological species is cultivated alone; and
purifying the biomolecule from the fermentation biomass.

2. The method of claim 1 wherein the isolated first biological species and the isolated second biological species are bacteria, fungi, algae or viruses.

3. The method of claim 1 wherein the growth medium is a nutrient medium.

4. The method of claim 1 wherein the step of purifying the biomolecule from the fermentation biomass is performed via vertical gas mediated extraction.
